(19) United States
(12) Patent Application Publication
(10) Pub. No.: US 2024/0316220 A1
(43) Pub. Date: Sep. 26, 2024

(54) CATALYST-FREE RADIOPAQUE MEDICAL HYDROGELS AND SYSTEMS FOR FORMING SAME

(71) Applicant: Boston Scientific Scimed, Inc., Maple Grove, MN (US)

(72) Inventors: Yen-Hao Hsu, Shrewsbury, MA (US); Joseph Thomas Delaney, JR., Minneapolis, MN (US); Cristian Parisi, Boston, MA (US)

(73) Assignee: Boston Scientific Scimed, Inc., Maple Grove, MN (US)

(21) Appl. No.: 18/614,277

(22) Filed: Mar. 22, 2024

Related U.S. Application Data
(60) Provisional application No. 63/492,067, filed on Mar. 24, 2023.

(51) Int. Cl. A61K 49/04; A61K 49/00; A61M 5/19

(52) U.S. Cl. CPC A61K 49/0442; A61K 49/0073; A61K 49/0438; A61M 5/19

(57) ABSTRACT

In some aspects, the present disclosure pertains to systems for forming a crosslinked polymer composition, the systems comprising (a) an iodinated multifunctional compound comprising either a plurality of electron-rich dienophile-containing moieties or a plurality of electron-poor diene-containing moieties and (b) a multifunctional multi-arm polymer comprising either a plurality of electron-poor diene-containing moieties in the case where the iodinated multifunctional compound comprises a plurality of electron-rich dienophile-containing moieties or a plurality of electron-rich dienophile-containing moieties in the case where the iodinated multifunctional compound comprises a plurality of electron-poor diene-containing moieties, wherein the crosslinked polymer composition is formed by a cycloaddition reaction occurring between the iodinated multifunctional compound and the multifunctional multi-arm polymer. Other aspects of the present disclosure pertain to crosslinked networks formed by such systems and to methods of treatment using such systems.

### CROSS-REFERENCE TO RELATED APPLICATIONS

This application claims the benefit of U.S. Provisional Patent Application Ser. No. 63/492,067 filed on Mar. 24, 2023, the disclosure of which is incorporated herein by reference.

### FIELD

The present disclosure relates to catalyst-free radiopaque hydrogels and to crosslinkable systems for forming catalyst-free radiopaque hydrogels, among other aspects. The catalyst-free radiopaque hydrogels and crosslinkable systems for forming the same are useful, for example, in various medical applications.

### BACKGROUND

In vivo crosslinked hydrogels based on star-poly(ethylene glycol) (star-PEG) polymers functionalized with reactive ester end groups which are reacted with lysine trimer (Lys-Lys-Lys) as a crosslinker to rapidly form crosslinked hydrogels, such as SpaceOAR®, have become clinically significant materials as adjuvants in radiotherapies. See “Augmenix Announces Positive Three-year SpaceOAR® Clinical Trial Results,” Imaging Technology News, Oct. 27, 2016.

Hydrogels in which some of the star-PEG branches have been functionalized, with 2,3,5-triiodobenzamide (TIB) groups replacing part of ester end groups, such as SpaceOAR® Vue®, have also been developed, which provide enhanced radiocontrast. See “Augmenix Receives FDA Clearance to Market its TraceIT® Tissue Marker,” BusinessWire Jan. 28, 2013. TraceIT® hydrogel remains stable and visible in tissue for three months, long enough for radiotherapy, after which it is absorbed and cleared from the body. Id.

While the above approach is effectual, using the arms of the star polymer to functionalize the hydrogel with iodine means that there are fewer arms available to crosslink. This can be overcome by adding more polymer, but the loading of solids increases, which can adversely impact viscosity. Reducing the molecular weight can cut down on the loading of solids, but this also can result in a lower melting point, and problems with processability. An additional effect of the reduced crosslink density per star polymer is that the resulting gel has a slower cure rate, which means the gel is liquid and mobile in vivo for longer time periods, opening up opportunities for unintended side-reactions and material displacement. Moreover, TIB is sparingly water soluble, meaning that there is an upper limit to how much iodine can be added before the solubility of the gel becomes impacted and it becomes difficult to form a smooth and consistent hydrogel. In the event that the concentration of TIB groups becomes so high that the star-PEG precipitates out of solution, the TIB groups can physically crosslink the system before reacting, requiring greater force to dispense. Furthermore, trilysine is utilized as a crosslinker to form a crosslinked hydrogel with TIB-functionalized star PEG. Unfortunately, amine-based biofluid is ubiquitous in vivo and acts as a natural source of crosslinker that competes with the trilysine, resulting in off-target crosslinking with less density in some cases. Finally, a buffer solution is added to the hydrogel precursor to maintain the pH value and avoid uncontrolled crosslinking conditions before injection, which increases the overall complexity of troubleshooting, manufacturing, and quality control.

For these and other reasons, alternative strategies for forming iodine-labelled crosslinked hydrogels that provide high reactive selectivity without the need for any buffer solution are desired.

### SUMMARY

The present disclosure provides alternative approaches to that described above based on a cycloaddition reaction.

In some aspects, the present disclosure provides a system for forming a crosslinked polymer composition, the system comprising (a) an iodinated multifunctional compound comprising either a plurality of electron-rich dienophile-containing moieties or a plurality of electron-poor diene-containing moieties and (b) a multifunctional multi-arm polymer comprising either a plurality of electron-poor diene-containing moieties in the case where the iodinated multifunctional compound comprises a plurality of electron-rich dienophile-containing moieties or a plurality of electron-rich dienophile-containing moieties in the case where the iodinated multifunctional compound comprises a plurality of electron-poor diene-containing moieties, wherein the crosslinked polymer composition is formed by a cycloaddition reaction occurring between the iodinated multifunctional compound and the multifunctional multi-arm polymer.

In some embodiments, which can be used in conjunction with the above aspects, the multifunctional multi-arm polymer comprises a plurality of hydrophilic polymer arms. Examples of hydrophilic polymer arms includes those that comprise one or more hydrophilic monomers selected from ethylene oxide, propylene oxide, N-vinyl pyrrolidone, an oxazoline monomer, hydroxyethyl acrylate, hydroxyethyl methacrylate, methyl ether acrylate, methyl ether methacrylate, acrylamide, methacrylamide, N-isopropylacrylamide, or a saccharide monomer.

In some embodiments, which can be used in conjunction with the above aspects and embodiments, an electron-rich dienophile-containing moiety or an electron-poor diene-containing moiety is linked to an end of each of two or more hydrophilic polymer arms. In some of these embodiments, the electron-rich dienophile-containing moiety or the electron-poor diene-containing moiety is linked to the end of each of the two or more hydrophilic polymer arms through a hydrolysable ester group.

In some embodiments, which can be used in conjunction with the above aspects and embodiments, a norbornenyl-group-containing moiety or a tetrazinyl-group-containing moiety is linked to an end of each of two or more hydrophilic polymer arms. In some of these embodiments, the norbornenyl-group-containing moiety or the tetrazinyl-group-containing moiety is linked to the end of each of the two or more hydrophilic polymer arms through a hydrolysable ester group.

In some embodiments, which can be used in conjunction with the above aspects and embodiments, the iodinated multifunctional compound comprises a monocyclic or multicyclic aromatic structure that is substituted with (a) one or more iodine groups and (b) either a plurality of electron-rich dienophile-containing moieties or a plurality of electron-poor diene-containing moieties. In some of these embodiments, the plurality of electron-rich dienophile-containing moieties or the plurality of electron-poor diene-containing moieties is linked to the aromatic structure though a hydrolysable ester group.

In some embodiments, which can be used in conjunction with the above aspects and embodiments, the iodinated multifunctional compound comprises a monocyclic or multicyclic aromatic structure that is substituted with (a) one or more iodine groups and (b) either a plurality of norbornenyl-group-containing moieties or a plurality of tetrazinyl-group-containing moieties. In some of these embodiments, the plurality of norbornenyl-group-containing moieties or the plurality of tetrazinyl-group-containing moieties is linked to the aromatic structure though a hydrolysable ester group.

In some embodiments, which can be used in conjunction with the above aspects and embodiments, system comprises a first composition that comprises the iodinated multifunctional compound in a first container and a second composition that comprises the multifunctional multi-arm polymer in a second container. In some of these embodiments, the first and second containers are independently selected from vials and syringe barrels. In some of these embodiments, the system further comprises a delivery device.

In other aspects, the present disclosure pertains to crosslinked networks formed by crosslinking an iodinated multifunctional compound in accordance with any of the above embodiments and the multifunctional multi-arm polymer in accordance with any of the above embodiments in a cycloaddition reaction.

In some embodiments, which can be used in conjunction with the above aspects, the crosslinked network has a radiopacity that is greater than 250 Hounsfield units.

In some embodiments, which can be used in conjunction with the above aspects and embodiments, the crosslinked network is a hydrogel.

In some embodiments, which can be used in conjunction with the above aspects and embodiments, the crosslinked network is in the form of particles having an average particle size ranging from 50 to 950 microns.

In other aspects, the present disclosure pertains to methods of treatment comprising administering to a subject a mixture that comprises an iodinated multifunctional compound in accordance with any of the above embodiments and a multifunctional multi-arm polymer in accordance with any of the above embodiments, wherein the crosslinked polymer composition is formed by a cycloaddition reaction occurring between the iodinated multifunctional compound and the multifunctional multi-arm polymer after administration.

Potential benefits associated with the present disclosure include one or more of the following: radiocontrast is maintained, highly selective crosslinking may be achieved thereby minimizing off-target crosslinking, buffer solutions may be avoided, the melting point of the solid components of the hydrogel can be maintained above 40° C. improving storage and handling, homogeneity of the final hydrogel may be improved, in vivo persistence may be obtained, and cure kinetics may be maintained.

The above and other aspects, embodiments, features and benefits of the present disclosure will be readily apparent from the following detailed description.

### BRIEF DESCRIPTION OF THE DRAWINGS

FIG. 1 schematically illustrates a method of forming a multifunctional multi-arm polymer that comprises electron-poor diene-containing moieties at the ends of the polymer arms, in accordance with an embodiment of the present disclosure.

FIG. 2 schematically illustrates a method of forming an iodinated multifunctional compound that comprises a plurality of electron-rich dienophile-containing moieties, in accordance with an embodiment of the present disclosure.

FIG. 3 schematically illustrates a method whereby the multifunctional multi-arm polymer product of FIG. 1 is crosslinked with the iodinated multifunctional compound of FIG. 2, according to an aspect of the present disclosure.

FIG. 4 schematically illustrates a method of forming an iodinated multifunctional compound that comprises a plurality of hydrophilic polymer segments terminated with electron-rich dienophile-containing moieties, in accordance with an embodiment of the present disclosure.

FIG. 5 schematically illustrates a multifunctional multi-arm polymer that comprises electron-rich dienophile-containing moieties at the ends of the polymer arms, in accordance with an embodiment of the present disclosure.

FIG. 6 schematically illustrates an iodinated multifunctional compound that comprises a plurality of electron-poor diene-containing moieties, in accordance with an embodiment of the present disclosure.

FIG. 7 illustrates a delivery device, in accordance with an embodiment of the present disclosure.

### DETAILED DESCRIPTION

In some aspects, the present disclosure pertains to compositions that comprise (a) a cycloaddition reaction product of a multifunctional multi-arm polymer that comprises a plurality of electron-poor diene-containing moieties and an iodinated multifunctional compound that comprises a plurality of electron-rich dienophile-containing moieties or (b) a cycloaddition reaction product of a multifunctional multi-arm polymer that comprises a plurality of electron-rich dienophile-containing moieties and an iodinated multifunctional compound that comprises a plurality of electron-poor diene-containing moieties.

The electron-poor diene-containing moieties may be, for example, tetrazinyl-group-containing moieties or 1,2,3-triazinyl group-containing moieties, among others. The electron-rich dienophile-containing moieties may be, for example, norbornenyl-group-containing moieties, electron-rich internal alkyne moieties, electron-rich alkene moieties, or electron-rich imine moieties, among others. Cycloaddition reactions between electron-poor diene-containing moieties and electron-rich dienophile-containing moieties are also sometimes referred to as inverse electron-demand Diels-Alder reactions.

Multifunctional multi-arm polymers that comprise a plurality of electron-poor diene-containing moieties include those that comprise a plurality of polymer arms (e.g., having two, three, four, five, six, seven, eight, nine, ten, eleven, twelve or more arms), wherein two or more polymer arms of the multifunctional multi-arm polymers each comprise one or more electron-poor diene moieties. In some embodiments, all of the arms of the multifunctional multi-arm polymers comprise one or more electron-poor diene-containing moieties.

Examples of electron-poor diene moieties include electron-poor diene-containing moieties that comprise tetrazinyl groups, specifically, 1,2,4,5-tetrazin-3-yl groups,

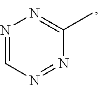

and 1,2,3-triazin-5-yl groups,

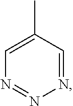

among others.

The moieties that comprise tetrazinyl groups may be linked to the polymer arms directly or through any suitable linking moiety, including linking moieties that comprise ester groups, amide groups, ether groups, amine groups, or carbonate groups, among others.

In various embodiments, the polymer arms are hydrophilic polymer arms. Such hydrophilic polymer arms may be composed of any of a variety of synthetic, natural, or hybrid synthetic-natural polymers including, for example, poly(alkylene oxides) such as poly(ethylene oxide) (PEO, also referred to as polyethylene glycol or PEG), poly(propylene oxide) (PPO) or poly(ethylene oxide-co-propylene oxide), poly(N-vinyl pyrrolidone), polyoxazolines including poly(2-alkyl-2-oxazolines) such as poly(2-methyl-2-oxazoline), poly(2-ethyl-2-oxazoline) and poly(2-propyl-2-oxazoline), poly(vinyl alcohol), poly(allyl alcohol), polyhydroxyethyl acrylate, polyhydroxyethyl methacrylate, PEG methyl ether acrylate or PEG methyl ether methacrylate, polyacrylamide, poly(N-isopropylacrylamide) (PNIPAAM), polysaccharides, including hyaluronic acid/hyaluronate and alginic acid/alginate, and combinations thereof. Such hydrophilic polymer arms may comprise one or more hydrophilic monomers selected from ethylene oxide, propylene oxide, N-vinyl pyrrolidone, an oxazoline monomer, hydroxyethyl acrylate, hydroxyethyl methacrylate, methyl ether acrylate, methyl ether methacrylate, acrylamide, methacrylamide, or N-isopropylacrylamide.

Typical average molecular weights for the multifunctional multi-arm polymers for use herein range from 15 to 50 kDa, among other values. In various embodiments, the multifunctional multi-arm polymers for use herein have a melting point of 40° C. or greater, preferably 45° C. or greater.

In some embodiments, the polymer arms extend from a core region. In certain of these embodiments, the core region comprises a residue of a polyol that is used to form the polymer arms. Illustrative polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols, such as glycerol, mannitol, sorbitol, inositol, xylitol, quebrachitol, threitol, arabitol, erythritol, pentaerythritol, adonitol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl)ethane, and 2,6-bis(hydroxyalkyl)cresols, among others.

In certain beneficial embodiments, the core region comprises a residue of a polyol that contains two, three, four, five, six, seven, eight, nine, ten or more hydroxyl groups. In certain beneficial embodiments, the core region comprises a residue of a polyol that is an oligomer of a sugar alcohol such as glycerol, mannitol, sorbitol, inositol, xylitol, erythritol, or pentaerythritol, among others.

In certain embodiments, the core region comprises a silsesquioxane. A silsesquioxane is a compound that has a cage-like silicon-oxygen core that is made up of Si—O—Si linkages and tetrahedral Si vertices. —H groups or exterior organic groups may be covalently attached to the cage-like silicon-oxygen core. In the present disclosure, when the core region comprises a silsesquioxane, the organic groups comprise polymer arms. Silsesquioxanes for use in the present disclosure include silsesquioxanes with 6 Si vertices, silsesquioxanes with 8 Si vertices, silsesquioxanes with 10 Si vertices, and silsesquioxanes with 12 Si vertices, which can act, respectively, as cores for 6-arm, 8-arm, 10-arm and 12-arm polymers. The silicon-oxygen cores are sometimes referred to as T₆, T₈, T₁₀, and T₁₂ cage-like silicon-oxygen cores, respectively (where T=the number of tetrahedral Si vertices). In all cases each Si atom is bonded to three O atoms, which in turn connect to other Si atoms. Silsesquioxanes include compounds of the chemical formula [RSiO_{3/2}]ₙ, where n is an integer of at least 6, commonly 6, 8, 10 or 12 (thereby having T₆, T₈, T₁₀ or T₁₂ cage-like silicon-oxygen core, respectively), and where R may be selected from an array of organic functional groups such as alkyl groups, aryl groups, alkoxyl groups, and polymeric arms, among others. The T₈ cage-like silicon-oxygen cores are widely studied and have the formula [RSiO_{3/2}]₈, or equivalently R₈Si₈O₁₂. Such a structure is shown here:

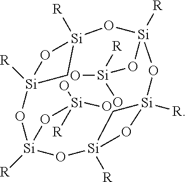

In the present disclosure, when the core region comprises a silsesquioxane, at least two R groups comprise polymer arms, and typically all R groups comprise polymer arms.

In certain embodiments, the electron-poor diene-containing moieties are linked to the polymer arms via hydrolysable ester groups.

Multifunctional multi-arm polymers having arms that comprise one or more electron-poor diene-containing moieties can be formed, for example, from multi-arm polymers having polymer arms that comprise one or more hydroxyl end groups and compounds that comprise an electron-poor diene-containing moiety and a carboxylic acid group, for example, a compound that comprises a tetrazinyl-group-containing moiety and a carboxylic acid group. For instance, an ester coupling reaction may be performed between the carboxylic acid group of a compound that comprises an electron-poor diene-containing moiety and a carboxylic acid group and hydroxyl groups of a multi-arm polymer having polymer arms that comprise one or more hydroxyl end groups. Such ester coupling reactions may be conducted using a suitable coupling reagent such as carbodiimide coupling reagent such as 1-(3-Dimethylaminopropyl)-N′-ethylcarbodiimide hydrochloride (EDC-HCl), dicyclohexylcarbodiimide (DCC) or diisopropylcarbodiimide (DIC), among others.

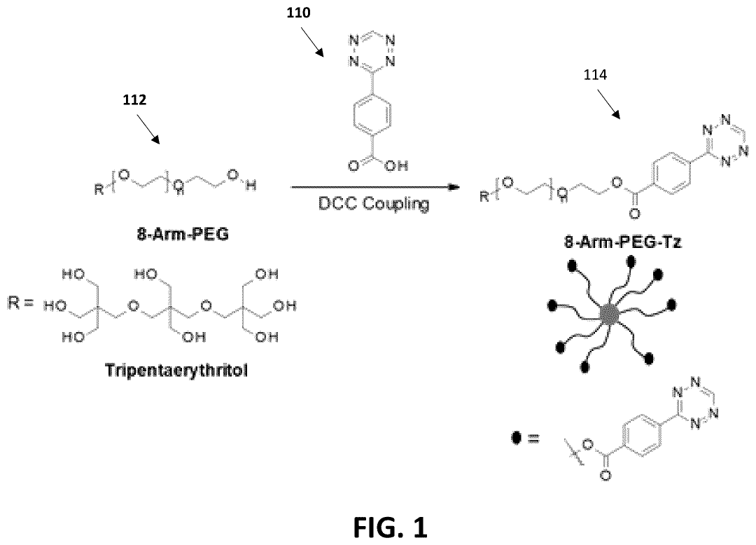

In a specific embodiment shown in FIG. 1, a compound that comprises a tetrazinyl-group-containing moiety, specifically, a 4-(1,2,4,5-tetrazin-3-yl)phenyl moiety,

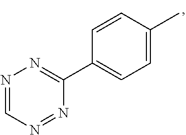

and a carboxylic acid group, in particular, 4-(1,2,4,5-tetrazin-3-yl) benzoic acid (CAS #1345866-65-0) (110), is reacted with a multi-arm polymer (112), which comprises a core region that comprises a polyol residue R, for example, a tripentaerythritol polyol residue, and 8 hydroxyl-terminated polyethylene oxide arms where n ranges from 30 to 140, in an ester coupling reaction utilizing dicyclohexylcarbodiimide (DCC) as a carbodiimide coupling reagent, to form a multi-arm polymer (114) having a core region and 8 polyethylene oxide arms, each terminating in a tetrazinyl-group-containing moiety that is linked to the polyethylene oxide arms by an ester group. The ester group is hydrolysable in vivo.

In other embodiments, multifunctional multi-arm polymers having arms that comprise one or more ester-linked, electron-poor diene-containing moieties can be formed from multi-arm polymers having polymer arms that comprise one or more carboxylic acid end groups and compounds that comprise an electron-poor diene-containing moiety and a hydroxyl group (e.g., 4-(1,2,4,5-tetrazin-3-yl) phenol, etc.).

Furthermore, multifunctional multi-arm polymers having arms that comprise one or more electron-poor diene-containing moieties can be formed (a) in an amide coupling reaction between a multi-arm polymer having polymer arms that comprise one or more amino end groups and a compound that comprises an electron-poor diene-containing moiety and a carboxylic acid group (e.g. 4-(1,2,4,5-tetrazin-3-yl) benzoic acid, etc.) or (b) in an amide coupling reaction between a multi-arm polymer having polymer arms that comprise one or more carboxylic acid end groups and a compound that comprises an electron-poor diene-containing moiety and an amino group (e.g. 4-(1,2,4,5-tetrazin-3-yl)phenyl)amine, 4-(1,2,4,5-tetrazin-3-yl)phenyl)methanamine, 4-(1,2,4,5-tetrazin-3-yl)phenyl)ethanamine, etc.).

Multifunctional multi-arm polymers such as those described above that comprise a plurality of electron-poor diene-containing moieties are useful, for example, in engaging in highly specific covalent bonding reactions with compounds that comprise ophile-containing moieties, including iodinated multifunctional compounds that comprise one or more iodine groups and a plurality of electron-rich dienophile-containing moieties as described hereinbelow. In various embodiments, compounds are employed that comprise two or more electron-rich dienophile moieties, in which case the reaction product may be a crosslinked reaction product. In some embodiments, the crosslinked reaction product contains absorbed water, in which case the crosslinked reaction product is a hydrogel.

Iodinated multifunctional compounds comprising one or more iodine groups and a plurality of electron-rich dienophile-containing moieties include, for example, iodinated multifunctional compounds containing one or more iodine groups and a plurality of norbornenyl-group-containing moieties, including, moieties containing 5-norbornyl groups,

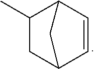

In particular embodiments, these iodinated multifunctional compounds may comprise a monocyclic or multicyclic aromatic structure, such as a benzene or a naphthalene structure, that is substituted with (a) one or more iodine groups and (b) a plurality of norbornenyl-group-containing moieties, which may be may be linked to the monocyclic or multicyclic aromatic structure directly or through any suitable linking moiety, including linking moieties that comprise ester groups, amide groups, ether groups, amine groups, or carbonate groups, among others.

Iodinated multifunctional compounds that comprise one or more iodine groups and a plurality of electron-rich dienophile-containing moieties may be formed, for example, from iodine-containing compounds that comprise a plurality of carboxyl groups and electron-rich dienophile-containing compounds that comprise an electron-rich dienophile-containing group and a hydroxyl group. For instance, an ester coupling reaction may be performed between a plurality of carboxylic acid groups of an iodine-containing compound that comprises a plurality of carboxylic acid groups and a hydroxyl group of electron-rich dienophile-containing compound that comprises an electron-rich dienophile-containing group and a hydroxyl group. The ester coupling reaction may be performed, for example, using a suitable coupling reagent such as carbodiimide coupling reagent, which may be selected from 1-(3-Dimethylaminopropyl)-N′-ethylcarbodiimide hydrochloride (EDC-HCl), dicyclohexylcarbodiimide (DCC) or diisopropylcarbodiimide (DIC), among others.

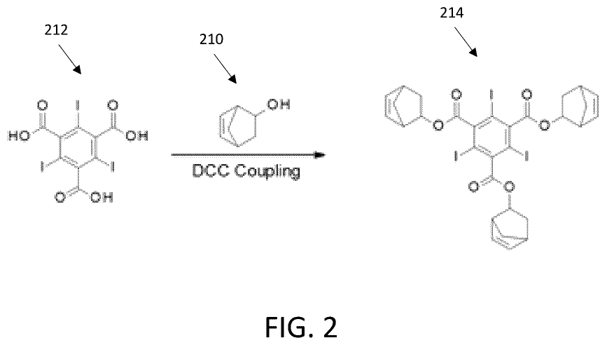

In a specific embodiment shown in FIG. 2, a hydroxyl-substituted norbornenyl compound, specifically, bicyclo[2.2.1]hept-5-en-ol (CAS #13080-90-5) (210), is reacted with an iodine-containing compound having three carboxylic acid groups, specifically, 2,4,6-triiodobenzene-1,3,5-carboxylic acid (CAS #79211-41-9) (212), in an ester coupling reaction utilizing dicyclohexylcarbodiimide (DCC) as a carbodiimide coupling reagent, to form a 1,3,5-norbornene-functionalized-2,4,6-triiodobenzene compound (214).

In other embodiments, iodinated multifunctional compounds that comprise one or more iodine groups and a plurality of electron-rich dienophile-containing moieties can be formed in an ester coupling reaction between iodine-containing compounds that comprise a plurality of hydroxyl groups (e.g., 2,4,6-triiodobenzene-1,3,5-triol, etc.) and electron-rich dienophile-containing compounds that comprise an electron-rich dienophile-containing group and a carboxylic acid group (e.g., bicyclo[2.2.1]hept-5-ene-2-carboxylic acid, etc.).

Furthermore, iodinated multifunctional compounds that comprise one or more iodine groups and a plurality of electron-rich dienophile-containing moieties can be formed (a) in an amide coupling reaction between iodine-containing compounds that comprise a plurality of amino groups (e.g., 2,4,6-triiodobenzene-1,3,5-triamine, 2,4,6-triiodobenzene-1,3,5-trimethanamine, 2,4,6-triiodobenzene-1,3,5-triethanamine, etc.) and electron-rich dienophile-containing compounds that comprise an electron-rich dienophile-containing group and a carboxylic acid group (e.g., bicyclo[2.2.1]hept-5-ene-2-carboxylic acid, etc.) or (b) in an amide coupling reaction between iodine-containing compounds that comprise a plurality of carboxylic acid groups (e.g., 2,4,6-triiodobenzene-1,3,5-tricarboxylic acid, etc.) and electron-rich dienophile-containing compounds that comprise an electron-rich dienophile-containing group and an amino group (e.g., bicyclo[2.2.1]hept-5-ene-2-amine, bicyclo[2.2.1]hept-5-ene-2-methamine, bicyclo[2.2.1]hept-5-ene-2-ethamine, etc.).

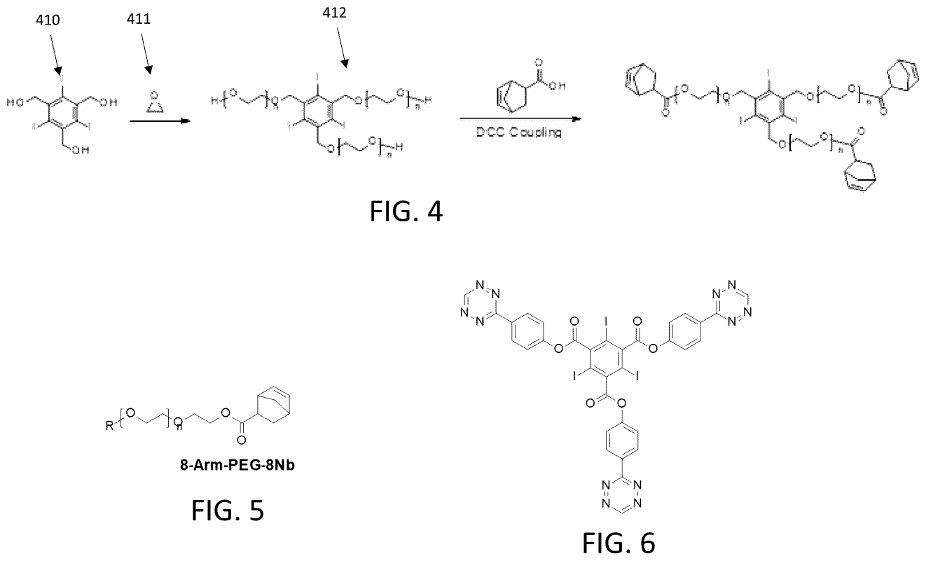

In addition, the degree of hydrophilicity of the iodinated multifunctional compounds that comprise one or more iodine groups and a plurality of electron-rich dienophile-containing moieties can be varied if desired. For example, with reference to FIG. 4, 1,3,5-triiodo-2,4,6-tris-hydroxymethylbenzene (CAS #178814-33-0) (410) may be used as an initiator to undergo ring-opening polymerization with ethylene oxide (411) forming a 1,3,5-triiodo-2,4,6-tris-polyethylene-oxide-functionalized compound (412) in which each polyethylene oxide (PEO) chain is terminated with a hydroxyl group. Compound (412) is further coupled with 5-norbornene-2-carboxylic acid (CAS #120-74-1) to obtain a 1,3,5-norbornene-functionalized-2,4,6-triiodobenzene compound (214), which comprises a benzene central structure substituted with three iodine groups and three norbornenyl-group-containing moieties, specifically, three norbornenyl-terminated PEO moieties. The number of repeating units (n) can be varied depending on the level of water solubility that is desired and may range, for example, ranging anywhere from 1 to 2 to 5 to 10 to 20 to 50 to 100 units (in other words, ranging between any two of the preceding numerical values), among other ranges.

When combined, an iodinated multifunctional compound comprising a plurality of electron-rich dienophile-containing moieties, for example, selected from those described above, among others, and a multifunctional multi-arm polymer that comprises a plurality of electron-poor diene-containing moieties, for example, selected from those described above, among others, will spontaneously and rapidly undergo a cycloaddition reaction thereby crosslinking the multifunctional multi-arm polymer and the iodinated multifunctional compound. This reaction will proceed at room temp and the rate will increase with increasing temperature above room temperature (e.g., at 37° C.). Such reactions can be conducted in vivo or ex vivo. The highly reactive selectivity of the cycloaddition reaction means that the reaction will only take place between the electron-rich dienophile groups (e.g., norbornenyl groups) and the electron-poor diene groups (e.g., tetrazinyl groups), thereby avoiding off-target or unintentional crosslinking in vivo.

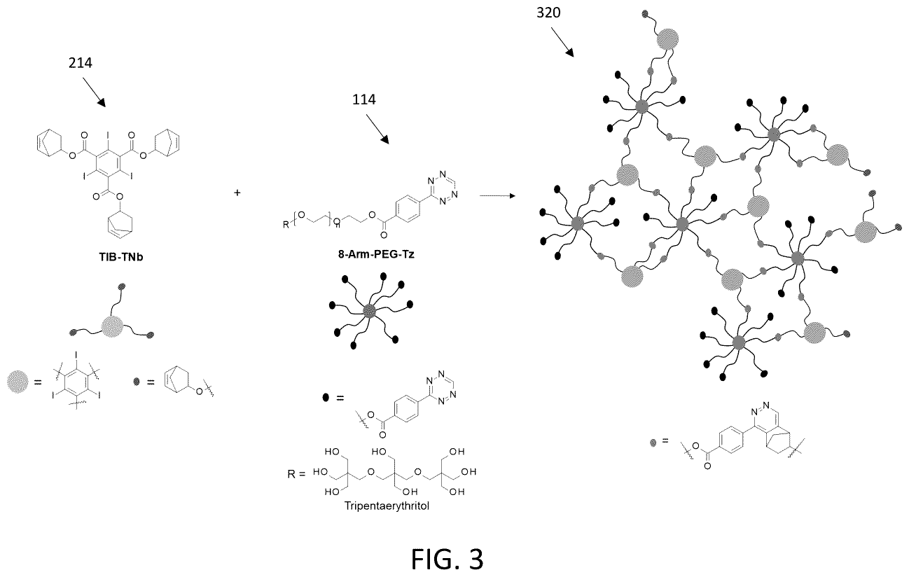

In a particular embodiment that is schematically represented in FIG. 3, the tetrazinyl-group-terminated multi-arm polymer (114) of FIG. 1 is coupled with the iodinated multifunctional norbornenyl-group-containing compound (214) of FIG. 2 via a cycloaddition reaction to form a crosslinked polymer composition (320) shown.

In other embodiments, analogous cycloaddition reactions can be performed by the reaction of multifunctional multi-arm polymers that comprise a plurality of electron-rich dienophile-containing moieties and iodinated multifunctional compounds comprising a plurality of electron-poor diene-containing moieties.

Multifunctional multi-arm polymers that comprise a plurality of electron-rich dienophile-containing moieties include those that comprise a plurality of polymer arms (e.g., having two, three, four, five, six, seven, eight, nine, ten, eleven, twelve or more arms), wherein two or more polymer arms of the multi-arm polymers each comprise one or more electron-rich dienophile moieties. In some embodiments, all of the arms of the multi-arm polymers comprise one or more electron-rich dienophile-containing moieties.

Examples of electron-rich dienophile-containing moieties include those that comprise norbornenyl groups. The moieties that comprise norbornenyl groups may be linked to the polymer arms directly or through any suitable linking moiety, including linking moieties that comprise ester groups, amide groups, ether groups, amine groups, or carbonate groups, among others.

In various embodiments, the polymer arms are hydrophilic polymer arms. Such hydrophilic polymer arms may be composed of any of a variety of synthetic, natural, or hybrid synthetic-natural polymers, including those described above, among others. In some embodiments, the polymer arms extend from a core region which may be selected from those described above, among others.

In certain embodiments, the electron-rich dienophile-containing moieties are linked to the polymer arms via hydrolysable ester groups.

Multifunctional multi-arm polymers having arms that comprise one or more electron-rich dienophile-containing moieties can be formed, for example, from multi-arm polymers having polymer arms that comprise one or more hydroxyl end groups and compounds that comprise an electron-rich dienophile-containing moiety and a carboxylic acid group, for example, a compound that comprises a norbornenyl-group-containing moiety and a carboxylic acid group. For instance, an ester coupling reaction may be performed between the carboxylic acid group of a compound that comprises an electron-rich dienophile-containing moiety (e.g., a norbornenyl-group-containing moiety) and a carboxylic acid group and hydroxyl groups of a multi-arm polymer having polymer arms that comprise one or more hydroxyl end groups. Such an ester coupling reaction may be conducted using a suitable coupling reagent, for example, a carbodiimide coupling reagent such as dicyclohexylcarbodiimide (DCC) or diisopropylcarbodiimide (DIC), among others.

In a specific embodiment, an electron-rich dienophile-containing compound that comprises an electron-rich dienophile-containing group and a carboxylic acid group (e.g., bicyclo[2.2.1]hept-5-ene-2-carboxylic acid, etc.) may be reacted with a multi-arm polymer in which each polymer arm comprises a terminal hydroxyl group. The reaction may be an ester coupling reaction utilizing dicyclohexylcarbodiimide (DCC) as a carbodiimide coupling reagent (or another reagent such as DIC, EDC-HCl, etc.), which forms a multifunctional multi-arm polymer (514) as shown in FIG. 5. The multifunctional multi-arm polymer (514) comprises a core region that comprises a polyol residue R, for example, a tripentaerythritol polyol residue, and 8 polyethylene oxide arms, where n ranges from 30 to 140. Each arm of the multifunctional multi-arm polymer (514) is terminated in a norbornenyl-group-containing moiety that is linked to the polyethylene oxide arm by an ester group.

In other embodiments, multifunctional multi-arm polymers having arms that comprise one or more electron-rich dienophile-containing moieties can be formed from multi-arm polymers having polymer arms that comprise one or more carboxylic acid end groups and compounds that comprise an electron-rich dienophile-containing moiety (e.g., a norbornenyl-group-containing moiety) and a hydroxyl group (e.g., bicyclo[2.2.1]hept-5-en-ol, etc.).

Furthermore, multifunctional multi-arm polymers having arms that comprise one or more electron-rich dienophile-containing moieties can be formed (a) in an amide coupling reaction between a multi-arm polymer having polymer arms that comprise one or more amino end groups and a compound that comprises an electron-rich dienophile-containing moiety (e.g., a norbornenyl-group-containing moiety) and a carboxylic acid group (e.g., bicyclo[2.2.1]hept-5-ene-2-carboxylic acid, etc.) or (b) in an amide coupling reaction between a multi-arm polymer having polymer arms that comprise one or more carboxylic acid end groups and a compound that comprises an electron-rich dienophile-containing moiety (e.g., a norbornenyl-group-containing moiety) and an amino group (e.g., bicyclo[2.2.1]hept-5-ene-2-amine, bicyclo[2.2.1]hept-5-ene-2-methamine, bicyclo[2.2.1]hept-5-ene-2-ethamine, etc.).

Multifunctional multi-arm polymers such as those described above that comprise a plurality of electron-rich dienophile-containing moieties are useful, for example, in engaging in highly specific covalent bonding reactions with compounds that comprise electron-poor diene-containing moieties, including iodinated multifunctional compounds that comprise one or more iodine groups and a plurality of electron-poor diene-containing moieties as described hereinbelow. In various embodiments, the compounds comprise two or more electron-poor diene moieties, in which case the reaction product may be a crosslinked reaction product. In some embodiments, the crosslinked reaction product contains absorbed water, in which case the crosslinked reaction product is a hydrogel.

Iodinated multifunctional compounds comprising one or more iodine groups and a plurality of electron-rich dienophile-containing moieties include, for example, iodinated multifunctional compounds containing one or more iodine groups and a plurality of tetrazinyl-group-containing moieties. In particular embodiments, these iodinated multifunctional compounds may comprise a monocyclic or multicyclic aromatic structure, such as a benzene or a naphthalene structure, that is substituted with (a) one or more iodine groups and (b) a plurality of tetrazinyl-group-containing moieties, which may be may be linked to the monocyclic or multicyclic aromatic structure directly or through any suitable linking moiety, including linking moieties that comprise ester groups, amide groups, ether groups, amine groups, or carbonate groups, among others.

Iodinated multifunctional compounds that comprise one or more iodine groups and a plurality of electron-poor diene-containing moieties may be formed, for example, from iodine-containing compounds that comprise a plurality of carboxyl groups and electron-poor diene-containing compounds that comprise an electron-poor diene-containing group and a hydroxyl group. For instance, an ester coupling reaction may be performed between a plurality of carboxylic acid groups of an iodine-containing compound that comprises a plurality of carboxylic acid groups and a hydroxyl group of electron-poor diene-containing compound that comprises an electron-poor diene-containing group and a hydroxyl group. The ester coupling reaction may be performed, for example, using a suitable coupling reagent such as carbodiimide coupling reagent, which may be selected from 1-(3-Dimethylaminopropyl)-N′-ethylcarbodiimide hydrochloride (EDC-HCl), dicyclohexylcarbodiimide (DCC) or diisopropylcarbodiimide (DIC), among others.

For example, a hydroxyl-substituted tetrazinyl compound (e.g., 4-(1,2,4,5-tetrazin-3-yl) phenol, etc.) may be reacted with an iodine-containing compounds having three carboxylic acid groups (e.g., 2,4,6-triiodobenzene-1,3,5-carboxylic acid, etc.) in an ester coupling reaction utilizing dicyclohexylcarbodiimide (DCC) as a carbodiimide coupling reagent, to form a 1,3,5-tetrazine-functionalized-2,4,6-triiodobenzene compound (614) as shown in FIG. 6.

In other embodiments, iodinated multifunctional compounds that comprise one or more iodine groups and a plurality of electron-poor diene-containing moieties can be formed in an ester coupling reaction between iodine-containing compounds that comprise a plurality of hydroxyl groups (e.g., 2,4,6-triiodobenzene-1,3,5-triol, 1,3,5-tris-hydroxymethyl-2,4,6-triiodobenzene, etc.) and electron-poor diene-containing compounds that comprise an electron-poor diene-containing group and a carboxylic acid group (e.g., 4-(1,2,4,5-tetrazin-3-yl) benzoic acid, etc.).

Furthermore, iodinated multifunctional compounds that comprise one or more iodine groups and a plurality of electron-poor diene-containing moieties can be formed (a) in an amide coupling reaction between iodine-containing compounds that comprise a plurality of amino groups (e.g., 2,4,6-triiodobenzene-1,3,5-triamine, 2,4,6-triiodobenzene-1,3,5-trimethanamine, 2,4,6-triiodobenzene-1,3,5-triethanamine, etc.) and electron-poor diene-containing compounds that comprise an electron-poor diene-containing group and a carboxylic acid group (e.g., 4-(1,2,4,5-tetrazin-3-yl) benzoic acid, etc.) or (b) in an amide coupling reaction between iodine-containing compounds that comprise a plurality of carboxylic acid groups (e.g., 2,4,6-triiodobenzene-1,3,5-tricarboxylic acid, etc.) and electron-poor diene-containing compounds that comprise an electron-poor diene-containing group and an amino group (e.g. 4-(1,2,4,5-tetrazin-3-yl)phenyl)amine, 4-(1,2,4,5-tetrazin-3-yl)phenyl)methanamine, 4-(1,2,4,5-tetrazin-3-yl)phenyl)ethanamine, etc.).

When combined, an iodinated multifunctional compound comprising a plurality of electron-poor diene-containing moieties, for example, selected from those described above, among others, and a multifunctional multi-arm polymer that comprises a plurality of electron-rich dienophile-containing moieties, for example, selected from those described above, among others, will spontaneously and rapidly undergo a cycloaddition reaction thereby crosslinking the multifunctional multi-arm polymer and the iodinated multifunctional compound. This reaction will proceed at room temp and the rate will increase with increasing temperature above room temperature (e.g., at 37° C.). Such reactions can be conducted in vivo or ex vivo. The high selectivity of the cycloaddition reaction means that the reaction will only take place between the electron-rich dienophile groups (e.g., norbornenyl groups) and the electron-poor diene groups (e.g. tetrazinyl groups), thereby avoiding off-target or unintentional crosslinking in vivo.

In various aspects, the present disclosure pertains to (a) crosslinked polymer compositions that comprise a cycloaddition reaction products of multifunctional multi-arm polymers that comprises a plurality of electron-poor diene-containing moieties and iodinated multifunctional compounds that comprise a plurality of electron-rich dienophile-containing moieties or (b) crosslinked polymer compositions that comprise a cycloaddition reaction products of multifunctional multi-arm polymers that comprises a plurality of electron-rich dienophile-containing moieties and iodinated multifunctional compounds that comprise a plurality of electron-poor diene-containing moieties. Various examples of such multifunctional multi-arm polymers and such iodinated multifunctional compounds are described above. In various embodiments, the crosslinked polymer compositions contain absorbed water in which case the crosslinked polymer compositions are hydrogels.

In various embodiments, such crosslinked polymer compositions are visible under fluoroscopy. In various embodiments, such crosslinked polymer compositions have a radiopacity that is greater than 100 Hounsfield units (HU), beneficially ranging anywhere from 100 HU to 50 HU to 500 HU to 750 HU to 1000 HU or more (in other words, ranging between any two of the preceding numerical values).

The crosslinked polymer compositions the present disclosure may be formed in vivo (e.g., using a delivery device like that described below), or the crosslinked polymer compositions may be formed ex vivo and subsequently administered to a subject. Such compositions can be used in a wide variety of biomedical applications, including medical devices, implants, and pharmaceutical compositions.

The crosslinked polymer compositions of the present disclosure may be in any desired form, including slabs, cylinders, coatings, and particles. In some embodiments, the crosslinked polymer compositions may be dried and then granulated into particles of suitable size. Granulating may be by any suitable process, for instance by grinding (including cryogrinding), crushing, milling, pounding, or the like. Sieving or other known techniques can be used to classify and fractionate the particles. Crosslinked polymer particles may vary widely in size, for example, typically having an average size ranging from 1 to 1000 microns, more typically from 50 to 950 microns.

In various embodiments, in addition to (a) a cycloaddition reaction product of a multifunctional multi-arm polymer that comprises a plurality of electron-poor diene-containing moieties and an iodinated multifunctional compound that comprises a plurality of electron-rich dienophile-containing moieties or (b) a cycloaddition reaction product of a multifunctional multi-arm polymer that comprises a plurality of electron-rich dienophile-containing moieties and an iodinated multifunctional compound that comprises a plurality of electron-poor diene-containing moieties, the crosslinked polymer compositions of the present disclosure may further include one or more additional agents. Such additional agents include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, anti-cancer drugs, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, and STING (stimulator of interferon genes) agonists, among others.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(II) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxyl or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, (e) imageable radioisotopes including ^{99m}Tc, ²⁰¹Th, ⁵¹Cr, ⁶⁷Ga, ⁶⁸Ga, ¹¹¹In, ⁶⁴Cu, ⁸⁹Zr, ⁵⁹Fe, ⁴²K, ⁸²Rb, ²⁴Na, ⁴⁵Ti, ⁴⁴Sc, ⁵¹Cr and ¹⁷⁷Lu, among others, and (f) radiocontrast agents such as metallic particles, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block copolymers, etc.), among others, and pH adjusting agents including various buffer solutes.

In various embodiments, kits are provided that include one or more delivery devices for delivering crosslinked particles to a subject. Such systems may include one or more of the following: a syringe barrel, which may or may not contain injectable crosslinked polymer particles as described hereinabove; a vial, which may or may not contain a crosslinked injectable particles as described hereinabove; a needle; a flexible tube (e.g., adapted to fluidly connect the needle to the syringe); and an injectable liquid such as water for injection, normal saline or phosphate buffered saline. Whether supplied in a syringe, vial, or other reservoir, the injectable crosslinked polymer particles may be provided in a form that is ready for injection (e.g., an injectable particle suspension) or in dry form (e.g., in powder form, from which an injectable particle suspension can be formed by added a suitable injectable liquid).

Where provided in an injectable form that contains crosslinked polymer particles, the crosslinked polymer compositions as described herein can be used for a number of purposes.

For example, such crosslinked polymer compositions can be injected to provide spacing between tissues, crosslinked polymer compositions can be injected (e.g., in the form of blebs) to provide fiducial markers, crosslinked polymer compositions can be injected for tissue augmentation or regeneration, crosslinked polymer compositions can be injected as a filler or replacement for soft tissue, crosslinked polymer compositions can be injected to provide mechanical support for compromised tissue, crosslinked polymer compositions be injected as a scaffold, and/or crosslinked polymer compositions can be injected as a carrier of therapeutic agents in the treatment of diseases and cancers and the repair and regeneration of tissue, among other uses.

After administration, the crosslinked polymer compositions of the present disclosure can be imaged using a suitable imaging technique.

As seen from the above, the crosslinked polymer compositions of the present disclosure may be used in a variety of medical procedures, including the following, among others: a procedure to implant a fiducial marker comprising crosslinked polymer particles, a procedure to implant a tissue regeneration scaffold comprising crosslinked polymer particles, a procedure to implant a tissue support comprising crosslinked polymer particles, a procedure to implant a tissue bulking agent comprising crosslinked polymer particles, a procedure to implant a therapeutic-agent-containing depot comprising crosslinked polymer particles, a tissue augmentation procedure comprising implanting crosslinked polymer particles, a procedure to introduce crosslinked polymer particles between a first tissue and a second tissue to space the first tissue from the second tissue.

The crosslinked polymer compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intra-vitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Crosslinked polymer compositions in accordance with the present disclosure include lubricious compositions for medical applications, compositions for therapeutic agent release (e.g., by including one or more therapeutic agents in a matrix of the crosslinked polymer), and implants (which may be formed ex vivo or in vivo) (e.g., compositions for use as tissue markers, compositions that act as spacers to reduce side effects of off-target radiation therapy, cosmetic compositions, etc.).

In further aspects, the present disclosure pertains to systems that can be used to form crosslinked polymer compositions. The systems may comprise (a) a first composition comprising an iodinated multifunctional compound comprising a plurality of electron-rich dienophile-containing moieties and a second composition comprising a multifunctional multi-arm polymer that comprises a plurality of electron-poor diene-containing moieties or (b) a first composition comprising an iodinated multifunctional compound comprising a plurality of electron-poor diene-containing moieties and a second composition comprising a multifunctional multi-arm polymer that comprises a plurality of electron-rich dienophile-containing moieties. Various examples of iodinated multifunctional compounds comprising electron-rich dienophile-containing moieties, iodinated multifunctional compounds comprising electron-poor diene-containing moieties, multifunctional multi-arm polymers comprising electron-rich dienophile-containing moieties, and multifunctional multi-arm polymers comprising electron-poor diene-containing moieties are described above.

The first and second compositions may be provided in first and second containers, respectively. For example, the first and second containers may be independently selected from vials and syringe barrels, among other formats.

In some aspects of the present disclosure, the systems are configured to dispense and combine the first and second compositions such that (a) the iodinated multifunctional compound, which may be either an iodinated multifunctional compound comprising electron-rich dienophile-containing moieties or an iodinated multifunctional compound comprising electron-poor diene-containing moieties, crosslinks via a cycloaddition reaction with (b) the multifunctional multi-arm polymer, which may be either a multifunctional multi-arm polymer comprising electron-poor diene-containing moieties (in the case wherein the iodinated multifunctional compound is an iodinated multifunctional compound comprising electron-rich dienophile-containing moieties) or a multifunctional multi-arm polymer comprising electron-rich dienophile-containing moieties (in the case where the iodinated multifunctional compound is an iodinated multifunctional compound comprising electron-poor diene-containing moieties).

Such systems are advantageous, for example, in that the cycloaddition reaction is highly selective, thereby minimizing off-target crosslinking. Such systems are also advantageous, for example, in that a buffer solution is not needed to maintain the pH at a particular value. Such systems are further advantageous, for example, in that iodine functionality, and thus radiopacity, is provided by the iodinated multifunctional compound that acts as a crosslinker for the multifunctional multi-arm polymer. This allows reactive end groups to be provided on each of the polymer arms, thereby maximizing the crosslinking capacity of the multifunctional multi-arm polymer, without sacrificing radiopacity.

The first composition may be a first fluid composition comprising the iodinated multifunctional compound or a first dry composition that comprises the iodinated multifunctional compound, to which a suitable fluid such as water for injection, saline, etc. can be added to form a first fluid composition. In addition to the iodinated multifunctional compound, the first composition may further comprise additional agents, including those described above.

The second composition may be a second fluid composition comprising the multifunctional multi-arm polymer or a second dry composition that comprises the multifunctional multi-arm polymer, to which a suitable fluid such as water for injection, saline, etc. can be added to form a second fluid composition. In addition to the multifunctional multi-arm polymer, the second composition may further comprise additional agents including as those described above.

In various embodiments, a system is provided that include one or more delivery devices for delivering first and second compositions to a subject.

In some embodiments, the system may include a delivery device that comprises a first reservoir that contains a first composition that comprises an iodinated multifunctional compound as described above and a second reservoir that contains a second composition that comprises a multifunctional multi-arm polymer as described above. During operation, the first composition and second composition are dispensed from the first and second reservoirs and combined, whereupon the iodinated multifunctional compound and the multifunctional multi-arm polymer combine and crosslink with one another to form a hydrogel.

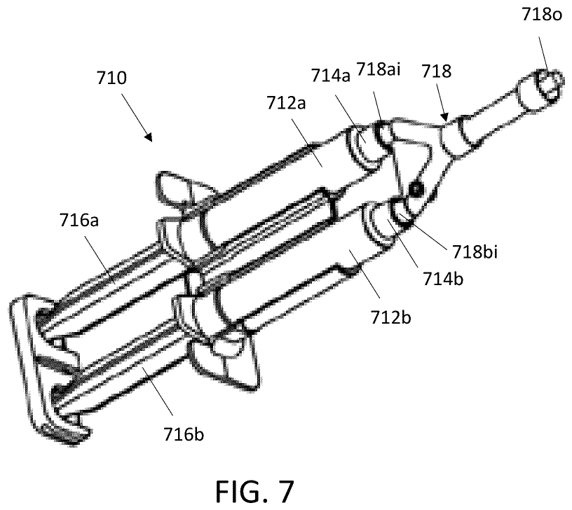

In particular embodiments, and with reference to FIG. 7, the system may include a delivery device 710 that comprises a double-barrel syringe, which includes first barrel 712a having a first barrel outlet 714a, which first barrel contains the first composition, a first plunger 716a that is movable in the first barrel 712a, a second barrel 712b having a second barrel outlet 714b, which second barrel 712b contains the second composition, and a second plunger 716b that is movable in the second barrel 712b. In some embodiments, the device 710 may further comprise a mixing section 718 having a first mixing section inlet 718ai in fluid communication with the first barrel outlet 714a, a second mixing section inlet 718bi in fluid communication with the second barrel outlet, and a mixing section outlet 7180.

In some embodiments, the device may further comprise a cannula or catheter tube that is configured to receive first and second fluid compositions from the first and second barrels. For example, a cannula or catheter tube may be configured to form a fluid connection with an outlet of a mixing section by attaching the cannula or catheter tube to an outlet of the mixing section, for example, via a suitable fluid connector such as a luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions interact and ultimately crosslink to form a hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture is initially in a fluid state and can be administered to a subject (e.g., a mammal, particularly, a human) by a variety of techniques. Alternatively, the first and second fluid compositions may be administered to a subject independently and a fluid admixture of the first and second fluid compositions formed on or in the subject. In either approach, a fluid admixture of the first and second fluid compositions is formed and used for various medical procedures.

For example, the first and second fluid compositions or a fluid admixture thereof can be injected to provide spacing between tissues, the first and second fluid compositions or a fluid admixture thereof can be injected (e.g., in the form of blebs) to provide fiducial markers, the first and second fluid compositions or a fluid admixture thereof can be injected for tissue augmentation or regeneration, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof be injected as a scaffold, and/or the first and second fluid compositions or a fluid admixture thereof can be injected as a carrier of therapeutic agents in the treatment of diseases and cancers and the repair and regeneration of tissue, among other uses.

After administration of the compositions of the present disclosure (either separately as first and second fluid compositions that mix in vivo or as a fluid admixture of the first and second fluid compositions) a crosslinked polymer composition, specifically, a crosslinked polymer hydrogel is ultimately formed at the administration location.

After administration, the compositions of the present disclosure can be imaged using a suitable imaging technique. Typically, the imaging techniques is an x-ray-based imaging technique, such as computerized tomography or X-ray fluoroscopy.

As seen from the above, the compositions of the present disclosure may be used in a variety of medical procedures, including the following, among others: a procedure to implant a fiducial marker comprising a crosslinked polymer composition formed from the first and second fluid compositions, a procedure to implant a tissue regeneration scaffold comprising a crosslinked polymer composition formed from the first and second fluid compositions, a procedure to implant a tissue support comprising a crosslinked polymer composition formed from the first and second fluid compositions, a procedure to implant a tissue bulking agent comprising a crosslinked polymer composition formed from the first and second fluid compositions, a procedure to implant a therapeutic-agent-containing depot comprising a crosslinked polymer composition formed from the first and second fluid compositions, a tissue augmentation procedure comprising implanting a crosslinked polymer composition formed from the first and second fluid compositions, and a procedure to introduce a crosslinked polymer composition formed from the first and second fluid compositions between a first tissue and a second tissue to space the first tissue from the second tissue.

The first and second fluid compositions or fluid admixtures of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Crosslinked polymer compositions in accordance with the present disclosure include lubricious compositions for medical applications, compositions for therapeutic agent release (e.g., by including one or more therapeutic agents in a matrix of the crosslinked polymer compositions), and implants (which may be formed ex vivo or in vivo) (e.g., compositions for use as tissue markers, compositions that act as spacers to reduce side effects of off-target radiation therapy, cosmetic compositions, etc.).

1. A system for forming a crosslinked polymer composition, the system comprising (a) an iodinated multifunctional compound comprising either a plurality of electron-rich dienophile-containing moieties or a plurality of electron-poor diene-containing moieties and (b) a multifunctional multi-arm polymer comprising either a plurality of electron-poor diene-containing moieties in the case where the iodinated multifunctional compound comprises a plurality of electron-rich dienophile-containing moieties or a plurality of electron-rich dienophile-containing moieties in the case where the iodinated multifunctional compound comprises a plurality of electron-poor diene-containing moieties, wherein the crosslinked polymer composition is formed by a cycloaddition reaction occurring between the iodinated multifunctional compound and the multifunctional multi-arm polymer.

2. The system of claim 1, wherein the multifunctional multi-arm polymer comprises a plurality of hydrophilic polymer arms.

3. The system of claim 2, wherein the hydrophilic polymer arms comprise one or more hydrophilic monomers selected from ethylene oxide, propylene oxide, N-vinyl pyrrolidone, an oxazoline monomer, hydroxyethyl acrylate, hydroxyethyl methacrylate, methyl ether acrylate, methyl ether methacrylate, acrylamide, methacrylamide, N-isopropylacrylamide, a saccharide monomer, and combinations thereof.

4. The system of claim 2, wherein an electron-rich dienophile-containing moiety or an electron-poor diene-containing moiety is linked to an end of each of two or more hydrophilic polymer arms of the plurality of hydrophilic polymer arms.

5. The system of claim 4, wherein the electron-rich dienophile-containing moiety or the electron-poor diene-containing moiety is linked to the end of each of the two or more hydrophilic polymer arms through a hydrolysable ester group.

6. The system of claim 2, wherein a norbornenyl-group-containing moiety or a tetrazinyl-group-containing moiety is linked to an end of each of two or more hydrophilic polymer arms of the plurality of hydrophilic polymer arms.

7. The system of claim 6, wherein the norbornenyl-group-containing moiety or the tetrazinyl-group-containing moiety is linked to the end of each of the two or more hydrophilic polymer arms through a hydrolysable ester group.

8. The system of claim 1, wherein the iodinated multifunctional compound comprises a monocyclic or multicyclic aromatic structure that is substituted with (a) one or more iodine groups and (b) either a plurality of electron-rich dienophile-containing moieties or a plurality of electron-poor diene-containing moieties.

9. The system of claim 8, wherein the plurality of electron-rich dienophile-containing moieties or the plurality of electron-poor diene-containing moieties is linked to the aromatic structure though a hydrolysable ester group.

10. The system of claim 1, wherein the iodinated multifunctional compound comprises a monocyclic or multicyclic aromatic structure that is substituted with (a) one or more iodine groups and (b) either a plurality of norbornenyl-group-containing moieties or a plurality of tetrazinyl-group-containing moieties.

11. The system of claim 10, wherein the plurality of norbornenyl-group-containing moieties or the plurality of tetrazinyl-group-containing moieties is linked to the aromatic structure though a hydrolysable ester group.

12. The system of claim 1, comprising a first composition that comprises the iodinated multifunctional compound in a first container and a second composition that comprises the multifunctional multi-arm polymer in a second container.

13. The system of claim 12, wherein the first and second containers are independently selected from vials and syringe barrels.

14. The system of claim 12, further comprising a delivery device.

15. A crosslinked network formed by crosslinking in a cycloaddition reaction (a) an iodinated multifunctional compound comprising either a plurality of electron-rich dienophile-containing moieties or a plurality of electron-poor diene-containing moieties and (b) a multifunctional multi-arm polymer comprising either a plurality of electron-poor diene-containing moieties in the case where the iodinated multifunctional compound comprises a plurality of electron-rich dienophile-containing moieties or a plurality of electron-rich dienophile-containing moieties in the case where the iodinated multifunctional compound comprises a plurality of electron-poor diene-containing moieties, wherein the crosslinked polymer composition is formed by a cycloaddition reaction occurring between the iodinated multifunctional compound and the multifunctional multi-arm polymer.

16. The crosslinked network of claim 15, wherein the crosslinked network has a radiopacity that is greater than 250 Hounsfield units.

17. The crosslinked network of claim 15, wherein the crosslinked network is a hydrogel.

18. The crosslinked network of claim 15, wherein the crosslinked network is in the form of particles having an average particle size ranging from 50 to 950 microns.

19. A method of treatment comprising administering to a subject a mixture that comprises (a) an iodinated multifunctional compound comprising either a plurality of electron-rich dienophile-containing moieties or a plurality of electron-poor diene-containing moieties and (b) a multifunctional multi-arm polymer comprising either a plurality of electron-poor diene-containing moieties in the case where the iodinated multifunctional compound comprises a plurality of electron-rich dienophile-containing moieties or a plurality of electron-rich dienophile-containing moieties in the case where the iodinated multifunctional compound comprises a plurality of electron-poor diene-containing moieties, wherein a crosslinked polymer composition is formed by a cycloaddition reaction occurring between the iodinated multifunctional compound and the multifunctional multi-arm polymer after administration.

20. The method of claim 19, wherein the mixture is administered to the patient via a double-barrel syringe.
